(19) United States
(12) Patent Application Publication
(10) Pub. No.: US 2008/0015573 A1
(43) Pub. Date: Jan. 17, 2008

(54) Medical device

(76) Inventor: Cino Rossi, Rome (IT)

Correspondence Address: WILLIAM COLLARD;COLLARD & ROE, P.C., 1077 NORTHERN BOULEVARD, ROSLYN, NY, 11576, US

(21) Appl. No.: 11/484,168

(22) Filed: Jul. 11, 2006

(51) Int. Cl. A61B 18/14

(52) U.S. Cl. 606/45; 606/49

(57) ABSTRACT

The medical device, suitable for cutting and/or coagulating portions of human and animal tissues, comprises sheath means; cutting means slidingly insertable into said sheath means and defining a proximal extremity and an opposing distal extremity, said cutting means further comprising cannula means which have longitudinal passage means and administration means for administration of substances, placed on said distal extremity.

The invention concerns a medical device, suitable for cutting portions of human and animal tissues.

In the execution of medical therapies, it may be necessary to cut excrescences of tissue located inside cavities of a human or animal body.

To execute these cuts, currently instruments are used that are known as electrosurgical unit which includes a machine that generates electricity with variable intensity and that is connected to a long thin metallic body, defined as the electrode, which is inserted in a sheath and which has a distal extremity preformed in the shape of a rigid loop that generally has the shape of a semicircle.

The electrode reaches the cutting point inside an endocavity by means of an endoscope, both flexible and rigid, which has been positioned inside the cavity in advance.

The endoscope is introduced inside the endocavity in such a manner that the distal extremity is located near a portion of tissue to be cut, for example an excrescence, so that the electrode's loop, which is guided by the endoscope, is able to reach it with precision.

Inside the endoscope, before inserting the electrode, a flexible needle is also inserted by sliding, which is connected to a device for injecting anesthetics such as, for example, a syringe, to anesthetize the area surrounding the portion of tissue to be cut.

Therefore, overall, to remove a portion of tissue, a doctor must insert in advance an endoscope inside of an endocavity; then he must insert the needle in the endoscope through a working channel provided for in it, so that its distal extremity is located near the point in which the cut must be made.

Then, by maneuvering the needle's proximal extremity which is purposefully kept outside the endoscope, the doctor inserts the tip of the needle in the tissue in the area to be anesthetized and injects the anesthetic in it.

When the injection is completed, the doctor removes the needle from the endoscope, while maintaining the latter in its initial operating position and then inserts the electrosurgical unit's electrode, until its distal extremity protrudes from the corresponding distal extremity of the endoscope.

The electrode's rigid tip in the shape of a loop is placed near the area of the tissue to be removed, by maneuvering the electrode from outside the endoscope.

After the loop has been correctly placed in position, the electrode will be powered with electricity at a set intensity which causes it to heat up to a temperature of approximately 300° C.; by maneuvering it properly, the doctor inserts the loop in the zone of the tissue to be removed, cutting off the portion of tissue that must be removed.

The doctor then modifies the electricity of the electrosurgical unit and adequately maneuvers the electrode to place its loop in contact with the cut blood vessels to coagulate and cauterize them.

This state of the art presents a drawback which is the need to use, besides the endoscope, two different instruments to cut portions of tissue, that is, a needle to anesthetize and an electrode to cut the portion of tissue and coagulate and cauterize the cut blood vessels.

A second drawback is that the doctor, while using two different instruments, must perform two maneuvers in succession, in order to insert and remove firstly from the endoscope the needle used to perform anesthesia and secondly in order to insert the electrosurgical unit's electrode to perform the cut and the cauterization of the cut tissue.

This state of the art requires a noticeable amount of time to insert and remove each time two different instruments inside the endoscope.

Furthermore, there is a risk, during the maneuver to insert and remove the needle for performing the anesthesia and the subsequent insertion of the electrode, that the endoscope may be moved away from the exact position at which the cut and removal must be carried out.

An object of the invention is to improve the state of the art.

Another object of the invention is to build a medical device that makes it possible to perform cuts and remove excrescences or other portions of tissue with a single instrument, thus eliminating the need to use two different instruments to perform anesthesia in advance and to subsequently cut a portion of tissues and coagulate and cauterize the cut blood vessels.

According to a first aspect of the invention it is provided a medical device, suitable for cutting and/or coagulating portions of human and animal tissues, comprising sheath means; cutting means slidingly insertable into said sheath means and defining a proximal extremity and an opposing distal extremity, characterized in that said cutting means comprises cannula means which have longitudinal passage means and administration means for administration of substances, placed on said distal extremity.

According to a second aspect of the invention it is provided a method for cutting and/or coagulating portions of human and animal tissues comprising: positioning sheath means near said portions of tissues; inserting and sliding cutting means into said sheath means for a first segment until a distal extremity of said cutting means provided with a tip, protrudes from said sheath means; heating said cutting means to activate them; cutting said portions characterized in that after said sliding for a first segment, injecting substances into said portions through said tip of said cutting means is provided for.

Therefore, the medical device and the method for cutting portions of human and animal tissues make it possible to inject substances, especially anesthetizing substances, and to cut portions of tissues by using a single instrument, thereby considerably reducing the operating time necessary to perform these procedures.

Further features and advantages will become clearer from the description of one embodiment of a medical device, suitable for cutting portions of human and animal tissues, illustrated by way of non-limitative example in the attached sheets of drawings in which:

Figures from 1a to 1d are enlarged, detailed and interrupted views of a distal portion of a medical device, suitable for cutting portions of human and animal tissues, in four consecutive steps of use;

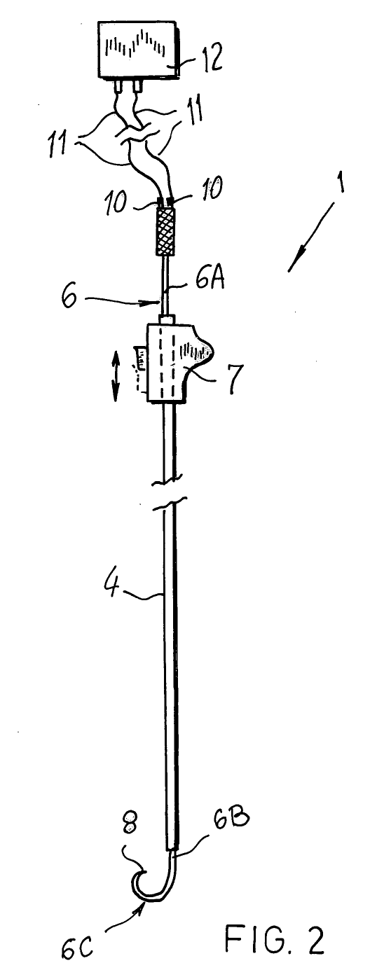

FIG. 2 is an overall view of a medical device, suitable for cutting portions of human and animal tissues;

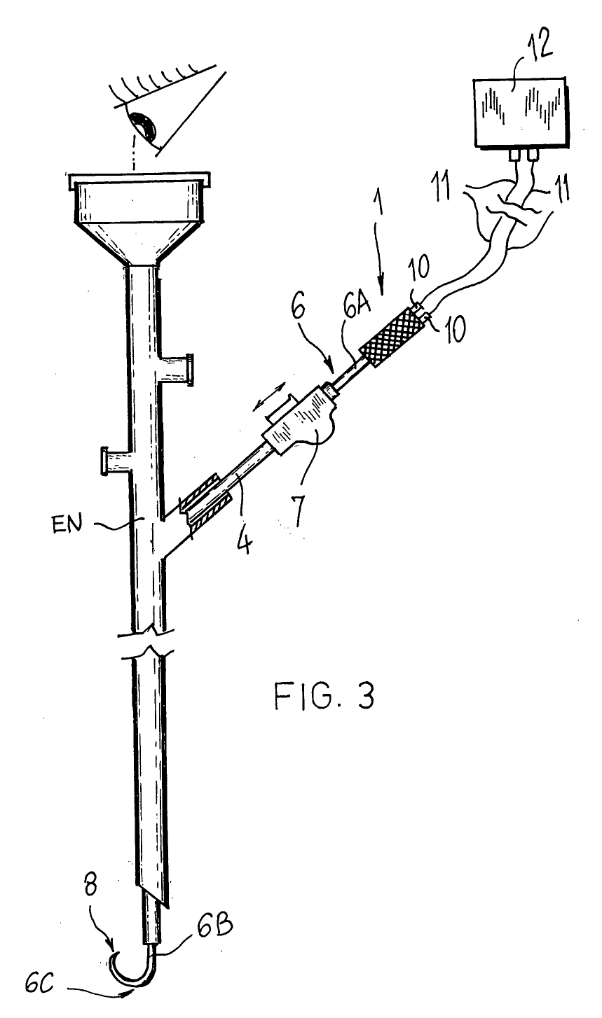

FIG. 3 is an interrupted longitudinal-section view of an endoscope inside which the medical device of FIG. 2, in a configuration of use is inserted.

With reference to FIG. 2, 1 indicates a medical device, suitable for cutting portions or excrescences 2 of human and/or animal tissues 3.

The medical device 1 comprises a flexible external sheath 4, through which a cavity 5 extends longitudinally in which can be inserted and moved by sliding a cannula 6, which is also flexible, and which is equipped with a handle 7 to allow the cannula 6 to be maneuvered and to slide the latter inside the external sheath 4 in a controlled manner.

The cannula 6 defines a proximal extremity 6A and an opposing distal extremity 6B which ends in a tip 8.

The cannula 6 is made of a flexible metallic material, specifically a material of the kind having a memory, i.e. that has the possibility to assume a configuration and to spontaneously return to said configuration after it is temporarily modified; also the cannula 6 is axially provided with a further cavity 9, which extends, on one end, up to the tip 8 and, on the other end, up to the proximal extremity 6A making the cannula 6 completely open for the administration of substances such as, for example, anesthetizing substances.

The distal extremity 6B of the cannula 6 defines an end segment which is shaped so as to form a loop 6C, designed to be placed near the excrescence 2 to be cut.

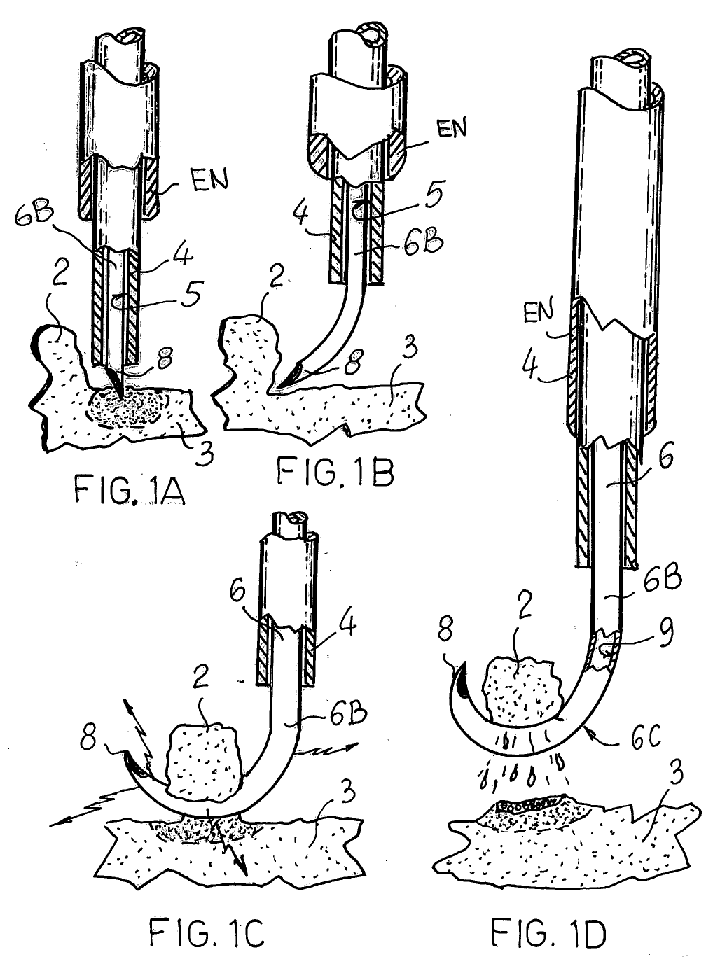

The shape of the loop 6C is maintained elastically straightened when the cannula 6 is inserted into the cavity 5 of the sheath 4, as can be seen in FIG. 1a.

When the cannula 6 is pushed toward the excrescence 2 to be cut, as illustrated in FIGS. 1b, 1c, and 1d, the end segment of the distal extremity 6B, protruding from the sheath 4, progressively regains its shape of a loop 6C, shaped and memorized in advance by the metallic material with which the cannula 6 is made of: this material comprises, for example, Nitinol.

The proximal extremity 6A of the cannula 6 is equipped with electrodes 10 (or with a connector) that can be connected by means of their respective cables 11, to an electric current generator 12 which, when the medical device 1 is in a suitable position to begin a cut, is activated to heat the cannula 6 to a temperature that will allow it, or more precisely will allow the loop 6C, to cut an excrescence 2 that protrudes from the tissue 3. After the cut is completed, the doctor modifies the intensity of the electric current supplied by the electric current generator 12 and, by maneuvering the cannula 6 through the proximal extremity 6A, places the loop 6C, in contact with the cut blood vessels, thereby cauterizing them.

The method for cutting portions of human and/or animal tissues comprises an initial step in which the sheath 4 and the cannula 6, which is inserted in its longitudinal cavity 5, are both positioned, by means of an endoscope “EN” that was placed in advance in an endocavity where a cut is required, so that the distal extremity 6B is located near an excrescence 2, or another portion of tissue, that is to be cut.

The doctor then uses the handle 7, causing the cannula 6 to slide into the cavity 5 of the sheath 4, pushing the tip 8 out of the sheath 4 for a first, short segment, so that the cannula 6 maintains substantially straight, or with a very limited curvature, its distal extremity 6B.

By keeping temporarily stationary this configuration between the sheath 4 and the cannula 6, the doctor introduces the tip 8 into the tissue 3, in an area surrounding the excrescence 2 to be cut and injects through the further cavity 9 and the tip 8 an anesthetizing substance, connecting, for example, the proximal extremity 6A of the cannula 6 to a syringe “S” that has been previously filled with this substance: in this way, the doctor administers local anesthesia.

Subsequently the doctor slightly pulls the sheath 4 toward the proximal extremity 6A, causing it to slide along the cannula 6 and uncovering the end segment of the distal extremity 6B.

Due to the memory of the metal (Nitinol) of which the cannula 6 is made, this distal extremity spontaneously regains the shape of a loop 6C which was given in advance to the distal extremity 6B.

In a subsequent step, the doctor places the loop 6C near the excrescence 2 to be cut and activates the electric current generator 12, causing the cannula 6 to be heated up to a temperature that allows, upon placing the loop 6C in contact with the excrescence 2, the latter to be cut.

Subsequently the doctor adjusts the intensity of the current to a suitable value and, by maneuvering the cannula 6 and its loop 6C, completes the operation by coagulating and cauterizing the cut blood vessels, by means of placing the loop 6C repeatedly in contact with them.

1) Medical device, medical device, suitable for cutting and/or coagulating portions of human and animal tissues, comprising sheath means; cutting means slidingly insertable into said sheath means and defining a proximal extremity and an opposing distal extremity, characterized in that said cutting means comprises cannula means which have longitudinal passage means and administration means for administration of substances, placed on said distal extremity.

2) Medical device according to claim 1, wherein said cannula means includes: a long thin metallic body made of heatable material; shaped means partially and/or completely wrappable around said portions, located at said distal extremity; connecting means for connecting said proximal extremity to a source of heating energy.

3) Medical device according to claim 2, wherein said heatable material is a flexible metallic material.

4) Medical device according to claim 3, wherein said flexible metallic material includes Nitinol.

5) Medical device according to claim 1, wherein said longitudinal passage means comprises a conduit that passes through said long thin metallic body and that has opposing extremities defining corresponding apertures that lead, respectively, to said distal extremity (6B) and to said proximal extremity.

6) Medical device according to claim 2, wherein said shaped means comprises a distal segment of said long thin metallic body, partially curved upon itself so that to shape a loop.

7) Medical device according to claim 2, wherein said shaped means includes a distal segment of said long, thin metallic body, completely curved upon itself so that to shape a substantially closed ring.

8) Medical device according to claim 1, wherein said administration means includes a tip that extends from said distal extremity, said tip having a longitudinally passage which extends from said conduit and is designed to be introduced into a portion of tissue into which must be administered a substance.

9) Method for cutting and/or coagulating portions of human and animal tissues comprising: positioning sheath means near said portions of tissues; inserting and sliding cutting means into said sheath means for a first segment until a distal extremity of said cutting means provided with a tip, protrudes from said sheath means; heating said cutting means to activate them; cutting said portions, characterized in that after said sliding for a first segment, injecting substances into said portions through said tip of said cutting means is provided for.

10) Method according to claim 9, wherein said substances comprise anesthetizing substances.

11) Method according to claim 9, wherein said cutting means includes thin cannula means equipped with a longitudinal cavity that extends through the entire length and that forms at a distal extremity a tip equipped with an opening toward the outside and connected to said longitudinal cavity.

12) Method according to claim 9, wherein after said injecting, protruding said distal extremity of said cannula from said sheath means for a further second segment beyond said initial segment, is provided for.

13) Method according to claim 9, wherein before said inserting, shaping said distal extremity of said cutting means according to a curved shape, thereby obtaining a shaped distal extremity, is provided for.

14) Method according to claim 13, wherein said shaped distal extremity shapes a loop curved according to a substantially semicircular profile.

15) Method according to claim 13, wherein said shaped distal extremity shapes a loop curved according to a substantially circular ring profile.

16) Method according to claim 12, wherein in which after said protruding, positioning said distal extremity in contact with said portions, is provided for.
